(19) United States
(12) Patent Application Publication
(10) Pub. No.: US 2025/0138012 A1
(43) Pub. Date: May 1, 2025

(54) BIOMARKER PANEL FOR DIAGNOSIS OR PREDICTION OF BRAIN METASTASIS OF LUNG CANCER, AND USE THEREOF

(71) Applicants: THE CATHOLIC UNIVERSITY OF KOREA INDUSTRY-ACADEMIC COOPERATION FOUNDATION, Seoul (KR); SAMSUNG LIFE PUBLIC WELFARE FOUNDATION, Seoul (KR)

(72) Inventors: Hae-Ock LEE, Seoul (KR); Myung-Ju AHN, Seoul (KR); Na Young KIM, Seoul (KR)

(21) Appl. No.: 18/837,365

(22) PCT Filed: Feb. 9, 2023
(86) PCT No.: PCT/KR2023/001915

§ 371 Date: Aug. 9, 2024

(30) Foreign Application Priority Data
Feb. 10, 2022 (KR) 10-2022-0017578

(51) Int. Cl. G01N 33/574; C12Q 1/6886

(52) U.S. Cl. CPC G01N 33/57423; C12Q 1/6886; C12Q 2600/156; G01N 2333/475; G01N 2333/90206; G01N 2333/91215; G01N 2333/916; G01N 2333/944; G01N 2333/948; G01N 2333/96433; G01N 2333/978; G01N 2333/99

(57) ABSTRACT

The present invention relates to a biomarker panel for the diagnosis or prediction of brain metastasis of lung cancer and a method for the diagnosis or prediction of metastatic brain tumors by using the panel. Accuracy and sensitivity in diagnosing lung cancer brain metastases were improved by providing a biomarker screened from pure tumor cells, beyond the limitations of biomarkers screened on the basis of bulk data of mixed tumor cells and cancer microenvironment cells.

### TECHNICAL FIELD

The present invention relates to a biomarker panel for the diagnosis or prediction of brain metastasis of lung cancer and a method for the diagnosis or prediction of brain metastasis using the panel.

### BACKGROUND ART

Lung cancer was a rare disease until the 19th century. However, as smoking became more common in the 20^{th} century, lung cancer began to increase rapidly, and the incidence of lung cancer is also rising rapidly in Korea. Also, because lung cancer is less treatable than other cancers, it is known to have the highest death rate among cancer patients although it does not have the highest incidence rate. Among patients developing lung cancer, it has been reported that some patients frequently metastasize to the brain. In particular, lung cancer has been reported to metastasize to the brain at a relatively high frequency in patients with non-small cell lung cancer, especially those developing adenocarcinoma (ADC), as well as in patients with small cell lung cancer, leading to the development of metastatic brain cancer. In the case where a patient develops metastatic brain cancer, the developed brain metastatic cancer may be treated through surgical resection, chemotherapy, radiation therapy, and the like. However, because it is reported that the incidence of side effects due to the treatment process of brain metastatic cancer is high, there are continuing efforts to prevent the onset of brain metastatic cancer as much as possible. In this way, it is expected that a method for the prevention of brain metastatic cancer can be developed through research into the causes and mechanisms of brain metastatic cancer.

Also, there is a need to identify genetic markers that enable appropriate treatment to be applied to patients suffering from lung cancer by diagnosing the brain metastasis of lung cancer or predicting the prognosis of lung cancer early on.

In related studies, to identify genetic changes associated with early distant metastasis in lung ADC, gene amplification and deletion in lung ADC with synchronous brain metastasis were compared with lung ADC with secondary brain metastasis. Among the several genetic changes, it has been reported that the amplification of ACTA2 is significantly associated with synchronous brain metastasis (HW Lee et al., Int J Oncol., 41:2013-20, 2012). ACTA2 is known to contribute to cell-derived mechanical stimulation and maintenance of cell shape and movement, and because cell motility is critically dependent on the actin cytoskeleton, the dynamics of cytoskeletal structures affected by the ACTA2 may be said to be essential for the invasion and metastasis of lung ADC (Fritz G. et al., CurrCancerDrugTargets, 6:1-14, 2006; Lambrechts A. et al., Int J BiochemCellBiol, 36:1890-909, 2004).

The present inventors invented the biomarker panel of the present invention by checking the expression patterns of various genes in patients who actually developed brain metastasis from lung cancer in order to screen biomarkers that can diagnose the possibility of brain metastasis of lung cancer and predict the prognosis of lung cancer.

### DISCLOSURE

### Technical Problem

The present invention is directed to providing a biomarker panel capable of diagnosing or predicting brain metastasis of lung cancer.

Also, the present invention is directed to providing a method for the screening of biomarkers capable of diagnosing brain metastasis of lung cancer from tumor cells having copy number variations through single-cell transcriptome analysis.

Further, the present invention is directed to providing a method for the diagnosis or prediction of brain metastasis of lung cancer using an agent capable of determining the levels of the biomarkers in a sample isolated from a patient.

### Technical Solution

The present invention provides a biomarker panel capable of diagnosing or predicting brain metastasis of lung cancer, which includes an agent for measuring the levels of two or more biomarkers selected from the group consisting of fibronectin 1 (FN1), macrophage migration inhibitory factor (MIF), perilipin 2 (PLIN2), Kruppel like factor 6 (KLF6), phosphofructokinase, platelet (PFKP), biliverdin reductase B (BLVRB), SRY-box transcription factor 4 (SOX4), glycogen phosphorylase L (PYGL), inositol monophosphatase 2 (IMPA2), vascular endothelial growth factor A (VEGFA), microsomal glutathione S-transferase 1 (MGST1), neugrin (NGRN), member RAS oncogene family (RAB11A), mortality factor 4 like 1 (MORF4L1), SMAD family member (SMAD9), lipoma HMGIC fusion partner L6 (LHFPL6), methenyltetrahydrofolate synthetase (MTHFS), mitochondrial ribosomal protein L18 (MRPL18), and peptidase D (PEPD).

In one embodiment, the biomarker panel capable of diagnosing or predicting brain metastasis of lung cancer may include an agent for measuring the levels of two or more biomarkers selected from the group consisting of fibronectin 1 (FN1), macrophage migration inhibitory factor (MIF), perilipin 2 (PLIN2), Kruppel like factor 6 (KLF6), phosphofructokinase, platelet (PFKP), biliverdin reductase B (BLVRB), SRY-box transcription factor 4 (SOX4), glycogen phosphorylase L (PYGL), inositol monophosphatase 2 (IMPA2), and vascular endothelial growth factor A (VEGFA). Specifically, the lung cancer may be lung adenocarcinoma.

In another embodiment, the biomarker panel capable of diagnosing or predicting brain metastasis of lung cancer may include an agent for measuring the levels of two or more biomarkers selected from the group consisting of microsomal glutathione S-transferase 1 (MGST1), neugrin (NGRN), member RAS oncogene family (RAB11A), mortality factor 4 like 1 (MORF4L1), SMAD family member (SMAD9), lipoma HMGIC fusion partner L6 (LHFPL6), methenyltetrahydrofolate synthetase (MTHFS), mitochondrial ribosomal protein L18 (MRPL18), and peptidase D (PEPD). Specifically, the lung cancer may be small-cell lung carcinoma (SCLC).

The term “biomarker panel” used in this specification refers to a panel including an arbitrary combination of a plurality of biomarkers for the diagnosis or prediction of brain metastasis of lung cancer, wherein the combination may be the entire set or any subset or subcombination thereof. That is, the biomarker panel may be a set of biomarkers and may also be a plurality of biomarkers in any form to be measured. For example, when FN1 is a part of the biomarker panel, either FN1 mRNA or FN1 protein may be considered to be a part of the panel. To increase the accuracy of diagnosis, a combination of multiple biomarkers may be more useful than a single biomarker. Specifically, detecting a plurality of biomarkers in a sample may increase the sensitivity and/or specificity of a test. Accordingly, in one embodiment, the biomarker panel may include two or more types of biomarkers. In other embodiments, the biomarker panel consists of the minimum number of biomarkers to generate the maximum amount of information. Accordingly, when the biomarker panel consists of “a set of biomarkers,” no biomarkers other than those used to form the set are included. In one embodiment, the biomarker panel may consist of two or more biomarkers among the biomarkers disclosed herein. In another embodiment, the biomarker panel may consist of three or more biomarkers among the biomarkers disclosed herein. In still another embodiment, the biomarker panel may consist of four or more biomarkers among the biomarkers disclosed herein. In yet another embodiment, the biomarker panel may consist of the 10 biomarkers disclosed herein in relation to the brain metastases of lung adenocarcinoma, and may consist of the 9 biomarkers disclosed herein in relation to the brain metastases of small-cell lung carcinoma. The biomarkers of the present invention show a statistically significant difference in the diagnosis of brain metastasis of lung cancer. In one embodiment, in a diagnostic test using these biomarkers alone or a combination of a plurality of the biomarkers, diagnostic accuracy may be improved with a sensitivity and specificity of approximately 85% or more, approximately 90% or more, approximately 95% or more, approximately 98% or more, and approximately 100%.

The agent for measuring the levels of the biomarkers may be a primer pair, a probe, or an antisense nucleotide. Specifically, the agent may be an agent for measuring the mRNA levels of biomarker genes, and may be a primer pair, a probe, or an antisense nucleotide that specifically binds to the genes. In one embodiment, each primer pair, probe, or antisense nucleotide may specifically bind to each of the biomarkers.

The agent for measuring the levels of the biomarkers may be an antibody. The antibody may be a monoclonal antibody, for example, a monoclonal antibody that specifically binds to any of the biomarkers. In one embodiment, the antibodies may specifically bind to each of the biomarkers.

Also, the present invention provides a method for the diagnosis or prediction of brain metastasis of lung cancer, which includes measuring the levels of two or more of the biomarkers in a sample isolated from a subject; and comparing the levels of the biomarkers with the corresponding results of the markers in a control sample.

In a specific embodiment, the method may be a method for the diagnosis or prediction of brain metastasis of lung adenocarcinoma, and a biomarker panel including two or more biomarkers selected from the group consisting of fibronectin 1 (FN1), macrophage migration inhibitory factor (MIF), perilipin 2 (PLIN2), Kruppel like factor 6 (KLF6), phosphofructokinase, platelet (PFKP), biliverdin reductase B (BLVRB), SRY-box transcription factor 4 (SOX4), glycogen phosphorylase L (PYGL), inositol monophosphatase 2 (IMPA2), and vascular endothelial growth factor A (VEGFA) may be used.

In another specific embodiment, the method may be a method for the diagnosis or prediction of brain metastasis of small-cell lung carcinoma, and a biomarker panel including two or more biomarkers selected from the group consisting of microsomal glutathione S-transferase 1 (MGST1), neugrin (NGRN), member RAS oncogene family (RAB11A), mortality factor 4 like 1 (MORF4L1), SMAD family member (SMAD9), lipoma HMGIC fusion partner L6 (LHFPL6), methenyltetrahydrofolate synthetase (MTHFS), mitochondrial ribosomal protein L18 (MRPL18), and peptidase D (PEPD) may be used.

The subject is a target for diagnosing brain metastasis of lung cancer, and, for example, is a target for predicting the possibility of metastasis, a target for diagnosing the status of metastasis, a target for determining the prognosis, a target for determining the dose of a drug for the prevention or treatment of brain metastasis, a target for determining a therapeutic method according to the progression of metastasis, and the like. The subject may be a vertebrate, specifically a mammal, an amphibian, a reptile, a bird, or the like. More specifically, the subject may be a mammal, for example, a human (Homo sapiens). The sample may include samples such as tissue, cells, whole blood, serum, plasma, saliva, sputum, cerebrospinal fluid, or urine isolated from the subject.

The levels of the biomarkers may be measured by measuring the mRNA levels or protein levels of the biomarker genes. Specifically, the mRNA level measurement is a process of confirming the presence and expression levels of mRNA of genes in a sample isolated from a subject in order to diagnose the brain metastasis, that is, a process of measuring the amount of mRNA. Analysis methods used for this purpose include a reverse transcription polymerase reaction (RT-PCR), a competitive reverse transcription polymerase reaction (competitive RT-PCR), a real-time reverse transcription polymerase reaction (real-time RT-PCR), an RNase protection assay (RPA), Northern blotting, DNA chips, and the like. Also, the protein level measurement is a process of confirming the presence and expression levels of the biomarker proteins in a sample of a subject in order to diagnose the brain metastasis. The amounts of proteins may be determined using an antibody that specifically binds to the biomarker proteins, and the protein expression level itself may be measured without using an antibody. The protein level measurement or comparative analysis methods include a protein chip assay, an immunoassay, a ligand binding assay, a matrix desorption/ionization time of flight mass spectrometry (MALDI-TOF) assay, a surface enhanced laser desorption/ionization time of flight mass spectrometry (SELDI-TOF) assay, a radioimmunoassay, a radioimmunodiffusion method, an Ouchterlony immunodiffusion method, rocket immunoelectrophoresis, immunohistologic staining, a complement fixation assay, a two-dimensional electrophoresis assay, liquid chromatography-mass spectrometry (LC-MS), liquid chromatography-mass spectrometry/mass spectrometry (LC-MS/MS), Western blot, an enzyme-linked immunosorbent assay (ELISA), and the like.

The method according to one embodiment includes comparing the levels of the biomarkers with the corresponding results of the corresponding biomarkers in a control sample. For example, when the biomarkers are overexpressed compared to the control sample, it is possible to determine that the brain metastasis of lung cancer has occurred or predict that the likelihood of brain metastasis is high. In a specific example, the expression levels of the biomarkers in lung cancer tissue were confirmed to be the same as the expression patterns of the biomarkers in metastatic brain tissue, indicating that metastatic brain cancer was caused by metastasis of cells derived from lung cancer tissue.

Also, the present inventors screened biomarkers that enable diagnosis of brain metastasis of lung cancer based on the differences in molecular characteristics of tumor cells according to the presence or absence of brain metastases through single-cell transcriptome analysis of cancer tissues of lung cancer patients in order to improve the accuracy and sensitivity of the biomarkers.

Accordingly, the present invention provides a method for screening biomarkers for the diagnosis or prediction of brain metastatic lung cancer, which includes:
performing single-cell transcriptome analysis on lung cancer samples isolated from a lung cancer patient with brain metastasis and a lung cancer patient without brain metastasis;
identifying tumor cells showing amplification of copy number variations (CNVs) through data obtained by the single-cell transcriptome analysis; and
screening proteins or genes encoding the same, which show a difference in expression between tumor cells derived from the lung cancer patient with brain metastasis and tumor cells derived from the lung cancer patient without brain metastasis at a single-cell level or pseudo-bulk level for the identified tumor cells.

The method for screening biomarkers may be further described with reference to FIG. 1.

Specifically, the identifying of the tumor cells may be performed by dividing cells into clusters based on the analyzed single-cell transcriptome data, classifying the cell types based on the expression levels of cell type-specific genes known in the art (Cell annotation), and identifying, as tumor cells, the cells showing the amplification of copy number variations among the cells belonging to the clusters showing gene expression in epithelial cells.

In the present invention, the accuracy and sensitivity in diagnosing brain metastasis of lung cancer may be improved beyond the limitations of the biomarkers screened based on the bulk data of mixed cancer microenvironment cells as well as existing tumor cells by comparing biomarkers that are screened by comparing the differences in expression of genes according to the brain metastasis in the identified pure tumor cells, and biomarkers that are screened based on the pseudo-bulk data of pure tumor cells in which the imbalance in the tumor ratio between patients is corrected, to screen the overlapping biomarkers.

The method may be applied to screen biomarkers for the diagnosis or prediction of metastasis of lung cancer, specifically lung adenocarcinoma or small-cell lung carcinoma.

### Advantageous Effects

Accuracy and sensitivity in diagnosing brain metastasis of lung cancer were improved by providing biomarkers screened from pure tumor cells, beyond the limitations of biomarkers screened on the basis of bulk data of mixed tumor cells and cancer microenvironment cells.

### DESCRIPTION OF DRAWINGS

FIG. 1 is a schematic diagram showing a process of screening biomarkers for diagnosis of brain metastasis of lung cancer according to the present invention.

FIG. 2A is a graph in which cell subtypes of tumor cells and normal epithelial cells are determined using single-cell transcriptome data of lung cancer patients according to the presence or absence of brain metastases, and FIG. 2B shows specific marker genes for each cell type.

FIG. 3 shows biomarkers screened through a combination of single tumor cell level analysis and pseudo-bulk level analysis. The biomarkers are screened from biomarkers having relatively high expression levels in lung cancer patients with brain metastasis.

FIG. 4 shows data confirming whether gene markers whose expression specifically increases in the lung tissue of lung cancer patients with brain metastasis are also expressed in a sample of brain tissue metastasized from lung adenocarcinoma.

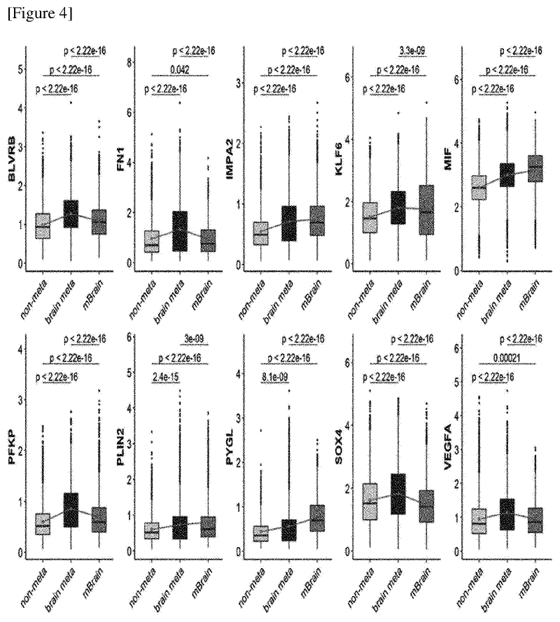

### MODE FOR INVENTION

Hereinafter, preferred embodiments are presented to aid in understanding the present invention. However, it should be understood that the following examples are provided only for easier understanding of the present invention, and are not intended to limit the scope of the present invention.

### EXAMPLES

### [Example 1] Screening of Biomarkers Through Single-Cell Transcriptome Analysis

### 1-1 Preparation of Lung Cancer Tissue Samples

This study was reviewed and approved by the Samsung Medical Center's Institutional Review Board (IRB) (IRB No. 2010-04-039-041), and 37 and 50 lung cancer tissue samples were obtained from 34 patients pathologically diagnosed with brain metastases of lung cancer without prior treatment and 43 lung cancer patients without brain metastasis, respectively. Specifically, metastatic lymph nodes and lung cancer tissues were collected from patients with advanced-stage lung cancer through bronchial ultrasound and bronchoscopy. Pleural fluid was obtained from lung cancer patients via a malignant pleural effusion fluid. On the day of surgery, a single-cell suspension was obtained using mechanical dissociation and enzymatic digestion. Thereafter, dead cells were removed by Ficoll-Paque PLUS (GE Healthcare, Sweden) separation.

### 1-2 Single-Cell RNA Sequencing and Pretreatment

3′ single-cell RNA sequencing was performed on a total of 5,000 cells from each cell suspension using a GemCode system (10× genomics, Pleasanton, CA, USA) according to the experimental protocol provided by the manufacturer. Readings of GemCode single-cell RNA sequencing were mapped to the GRCh38 human reference genome by a Cell Ranger toolkit (version 5.0.0). Quality measures of mitochondrial genes (less than 20%) and gene counts (greater than 200) calculated from a gene-cell-barcode matrix that did not undergo a standardization process were applied. Also, cells determined to be doublets were removed from the R package Scrublet toolkit (Samuel L. Wolock et al., 2019) in order to exclude cases where two or more cells were captured in one gem (doublet), (Samuel L. Wolock et al., 2019). The number of UMIs for genes in each cell was log-normalized to transcripts per million (TPM)-like values, and gene expression was then quantified on the scale of log (TPM+1). For sub-analyses, the gene expression was corrected by performing z-transformation, which calculates cell cycle scores based on typical cell cycle-related genes to apply regression analysis to mitigate the effect on the cell cycle scores.

### 1-3 Cell Clustering

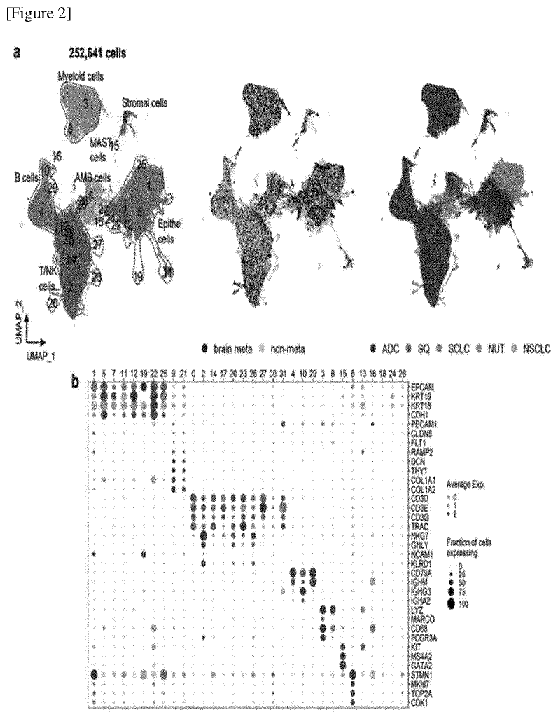

The single-cell RNA-sequencing results of Examples 1-2 were analyzed using unsupervised clustering, and the analysis results showed subclusters largely divided by tumor and surrounding immune environment regions (FIG. 2A). Specifically, variable genes were selected from the R package Seurat R toolkit (Tim Stuart et al., 2019) and used to calculate principal components (PCs). A subset of principal components (PC=40) for cell clustering was selected by the R function ElbowPlot in the Seurat package and used for UMAP visualization. The cell types of each cluster were defined and classified based on the expression levels of known marker genes (FIG. 2B).

### 1-4 Tumor Cell Screening

Among the above cell types, tumor cells showing amplification of gene copy number variation were identified among cells belonging to a cluster showing expression of epithelial cell markers. Specifically, for each sample, gene expression patterns of cells in the surrounding immune environments were used as the control using the R package CopyKAT toolkit (Ruli Gao et al., 2021) to predict copy number variations in epithelial cells. The parameters used in the calculation had the default values, but the minimum number of genes per chromosome (ngene.chr) for cell filtering was loosely adjusted to 3, and the segmentation parameters (KS.cut) were strictly adjusted to 0.05. Epithelial cells observed as aneuploids in the prediction results were screened as tumor cells.

### 1-5 Classification of Tumor Cells According to Type of Lung Cancer and Screening of Brain Metastasis-Specific Biomarkers

Secondary sample classification was performed according to the type of lung cancer to perform comparison of gene expression according to the onset of brain metastasis for lung adenocarcinoma and small-cell lung carcinoma. For screening of biomarkers, the log (fold change) (log FC) between the two groups (tumor cells derived from patients with brain metastasis vs. tumor cells derived from patients without brain metastasis) was calculated using the R function FindMarkers in the Seurat package. The significance of differences was determined by a Wilcoxon signed-rank test and Bonferroni correction. As the biomarkers for the diagnosis of brain metastasis at a single-cell level, genes whose proportion of cells with expression (pct) was greater than 0.25, FDR and P values were less than 0.01, and log FC was greater than 0.25, and genes whose proportion of samples with expression (pct) at a pseudo-bulk level was greater than 0.25, a P value was less than 0.01, and log FC was greater than 0.25 were selected.

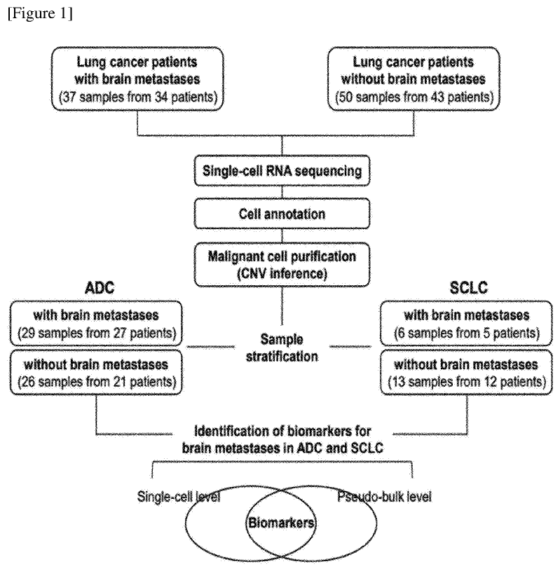

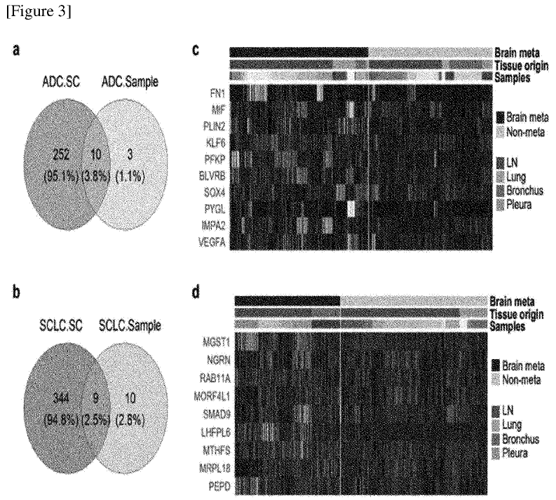

Specifically, the results of classifying the samples according to the type of lung cancer and whether or not brain metastasis occurred are shown in FIG. 1. For each of the tumor cell groups derived from lung adenocarcinoma and small-cell lung carcinoma, genes or proteins whose expression levels differed depending on whether brain metastasis occurred were screened as the biomarkers. As a result, 262 genes were screened as the biomarkers for the diagnosis of brain metastasis of lung adenocarcinoma, and 353 genes were screened as the biomarkers for the diagnosis of brain metastasis of small-cell lung carcinoma (FIG. 3A). The biomarkers were screened at the single-cell level and the tumor cells were simultaneously converted to the pseudo-bulk levels of the samples in order to screen the biomarkers. As a result, 13 and 19 genes were screened as the biomarkers for the diagnosis of brain metastasis of lung adenocarcinoma and small-cell lung carcinoma, respectively (FIG. 3B).

Finally, among the biomarkers screened at the single-cell level and the biomarkers screened at the pseudo-bulk level for the diagnosis of brain metastases of lung adenocarcinoma and small-cell lung carcinoma, 10 and 9 overlapping biomarkers were selected, respectively. The average relative expression values of the biomarkers for the diagnosis of brain metastasis of lung adenocarcinoma and small-cell lung carcinoma are shown in Tables 1 and 2 below, respectively.

TABLE 1

| | | | | | | | | | | UMI average | |
|---|---|---|---|---|---|---|---|---|---|---|---|
| | | | | | | | | | | expression levels | |
| | | | | | | | | | | Patients | Patients |
| Lung | Single-cell levels | | | | | Pseudo-bulk levels | | | | with | without |
| adenocarcinoma | | P- | | | | | P- | | | brain | brain |
| genes | log FC | value | FDR | pct. 1 | pct. 2 | log FC | value | pct. 1 | pct. 2 | metastasis | metastasis |
| FN1 | 0.683 | 0 | 0 | 0.438 | 0.294 | 0.763 | 0.007 | 0.966 | 0.88 | 7.9 | 3.9 |
| MIF | 0.384 | 0 | 0 | 0.997 | 0.991 | 0.301 | 0.009 | 1 | 1 | 87.4 | 40.7 |
| PLIN2 | 0.38 | 0 | 0 | 0.597 | 0.384 | 0.583 | 0 | 0.966 | 0.92 | 4 | 1 |
| KLF6 | 0.376 | 0 | 0 | 0.924 | 0.853 | 0.374 | 0.001 | 1 | 1 | 24.6 | 10.4 |
| PFKP | 0.373 | 0 | 0 | 0.653 | 0.445 | 0.281 | 0.009 | 1 | 0.96 | 4.8 | 1.1 |
| BLVRB | 0.362 | 0 | 0 | 0.899 | 0.815 | 0.321 | 0.003 | 0.966 | 0.96 | 10.9 | 4.1 |
| SOX4 | 0.305 | 0 | 0 | 0.928 | 0.856 | 0.458 | 0.004 | 0.966 | 0.92 | 24.8 | 14.6 |
| PYGL | 0.303 | 0 | 0 | 0.438 | 0.131 | 0.251 | 0.002 | 0.966 | 0.84 | 2 | 0.3 |
| IMPA2 | 0.298 | 0 | 0 | 0.677 | 0.435 | 0.254 | 0.003 | 0.966 | 0.88 | 3.7 | 1 |
| VEGFA | 0.295 | 0 | 0 | 0.822 | 0.677 | 0.296 | 0.006 | 0.966 | 0.92 | 8.9 | 4.5 |

TABLE 2

| | | | | | | | | | | UMI average | |
|---|---|---|---|---|---|---|---|---|---|---|---|
| | | | | | | | | | | expression levels | |
| Small-cell | | | | | | | | | | | Patients |
| lung | Single-cell levels | | | | | Pseudo-bulk levels | | | | Patients | without |
| carcinoma | | P- | | | | | P- | | | with brain | brain |
| genes | log FC | value | FDR | pct. 1 | pct. 2 | log FC | value | pct. 1 | pct. 2 | metastasis | metastasis |
| MGST1 | 0.692 | 0 | 0 | 0.749 | 0.415 | 0.572 | 0.01 | 1 | 1 | 6.9 | 1.6 |
| NGRN | 0.481 | 0 | 0 | 0.908 | 0.745 | 0.409 | 0.006 | 1 | 1 | 5.2 | 2.1 |
| RAB11A | 0.451 | 0 | 0 | 0.87 | 0.733 | 0.477 | 0.002 | 1 | 1 | 5.1 | 1.9 |
| MORF4L1 | 0.394 | 0 | 0 | 0.937 | 0.842 | 0.365 | 0.004 | 1 | 1 | 6.1 | 2.9 |
| SMAD9 | 0.323 | 0 | 0 | 0.659 | 0.393 | 0.397 | 0.01 | 1 | 1 | 1.8 | 0.8 |
| LHFPL6 | 0.322 | 0 | 0 | 0.419 | 0.082 | 0.399 | 0.002 | 1 | 1 | 0.9 | 0.1 |
| MTHFS | 0.286 | 0 | 0 | 0.605 | 0.372 | 0.285 | 0.001 | 1 | 1 | 1.6 | 0.6 |
| MRPL18 | 0.273 | 0 | 0 | 0.843 | 0.695 | 0.283 | 0.007 | 1 | 1 | 3.1 | 1.8 |
| PEPD | 0.26 | 0 | 0 | 0.571 | 0.465 | 0.289 | 0.004 | 1 | 1 | 2.5 | 0.8 |

### [Example 2] Verification and Diagnosis of Brain Metastasis of Lung Cancer Using Screened Biomarkers

The expression patterns of the selected biomarkers in tumor cells were compared in lung cancer tissue samples and metastatic brain cancer samples from lung cancer patients with brain metastasis. As a result, it was confirmed that the two samples showed the same biomarker expression pattern. These results demonstrate that metastatic brain cancer is caused by cells derived from lung cancer tissue, and at the same time, indicate that the screened biomarkers may be used to diagnose brain metastasis of lung cancer.

1. A biomarker panel for the diagnosis or prediction of brain metastasis of lung cancer, comprising an agent for measuring the levels of two or more biomarkers selected from the group consisting of fibronectin 1 (FN1), macrophage migration inhibitory factor (MIF), perilipin 2 (PLIN2), Kruppel like factor 6 (KLF6), phosphofructokinase, platelet (PFKP), biliverdin reductase B (BLVRB), SRY-box transcription factor 4 (SOX4), glycogen phosphorylase L (PYGL), inositol monophosphatase 2 (IMPA2), and vascular endothelial growth factor A (VEGFA).

2. A biomarker panel for the diagnosis or prediction of brain metastasis of lung cancer, comprising an agent for measuring the levels of two or more biomarkers selected from the group consisting of microsomal glutathione S-transferase 1 (MGST1), neugrin (NGRN), member RAS oncogene family (RAB11A), mortality factor 4 like 1 (MORF4L1), SMAD family member (SMAD9), lipoma HMGIC fusion partner L6 (LHFPL6), methenyltetrahydrofolate synthetase (MTHFS), mitochondrial ribosomal protein L18 (MRPL18), and peptidase D (PEPD).

3. The biomarker panel of claim 1, wherein the lung cancer is lung adenocarcinoma.

4. The biomarker panel of claim 2, wherein the lung cancer is small-cell lung carcinoma.

5. The biomarker panel of claim 1 or 2, wherein the agent for measuring the levels of biomarkers is a primer pair, a probe, or an antisense nucleotide.

6. A method of screening a biomarker for the diagnosis or prediction of brain metastatic lung cancer, comprising:
performing single-cell transcriptome analysis on lung cancer samples separated from a lung cancer patient with brain metastasis and a lung cancer patient without brain metastasis;
identifying tumor cells showing amplification of copy number variations (CNVs) through data obtained by the single-cell transcriptome analysis; and
screening proteins or genes encoding the same, which show a difference in expression between tumor cells derived from the lung cancer patient with brain metastasis and tumor cells derived from the lung cancer patient without brain metastasis at a single-cell level or pseudo-bulk level for the identified tumor cells.

7. The method of claim 6, wherein the overlapping proteins or genes encoding the same are screened as biomarkers by comparing the screened proteins or genes encoding the same at the single-cell level or pseudo-bulk level.

8. The method of claim 6, wherein the lung cancer is lung adenocarcinoma or small-cell lung carcinoma.

9. A method for the diagnosis or prediction of brain metastasis of lung cancer, comprising:
measuring the levels of two or more biomarkers selected from the group consisting of fibronectin 1 (FN1), macrophage migration inhibitory factor (MIF), perilipin 2 (PLIN2), Kruppel like factor 6 (KLF6), phosphofructokinase, platelet (PFKP), biliverdin reductase B (BLVRB), SRY-box transcription factor 4 (SOX4), glycogen phosphorylase L (PYGL), inositol monophosphatase 2 (IMPA2), and vascular endothelial growth factor A (VEGFA) in a sample isolated from a subject; and
comparing the levels of the biomarkers with the corresponding results of the markers in a control sample.

10. A method for the diagnosis or prediction of brain metastasis of lung cancer, comprising:
measuring the levels of two or more biomarkers selected from the group consisting of microsomal glutathione S-transferase 1 (MGST1), neugrin (NGRN), member RAS oncogene family (RAB11A), mortality factor 4 like 1 (MORF4L1), SMAD family member (SMAD9), lipoma HMGIC fusion partner L6 (LHFPL6), methenyltetrahydrofolate synthetase (MTHFS), mitochondrial ribosomal protein L18 (MRPL18), and peptidase D (PEPD) in a sample isolated from a subject; and
comparing the levels of the biomarkers with the corresponding results of the markers in a control sample.

11. The method of claim 9, wherein the lung cancer is lung adenocarcinoma.

12. The method of claim 10, wherein the lung cancer is small-cell lung carcinoma.

13. The method of claim 9 or 10, further comprising:
determining that the brain metastasis has occurred or the likelihood of brain metastasis is high when the relative expression levels of the two or more biomarkers are high compared to the control.
